(19) United States
(12) Patent Application Publication
(10) Pub. No.: US 2025/0287191 A1
(43) Pub. Date: Sep. 11, 2025

(54) METHOD, APPARATUS AND SYSTEM FOR PROVIDING SAFETY CARE SERVICE USING ARTIFICIAL INTELLIGENCE

(71) Applicant: Nectarsoft Co., Ltd., Seoul (KR)

(72) Inventors: Seong Ho PARK, Goyangi-si (KR); Myung Ju KANG, Seoul (KR); Eui Gu KIM, Seoul (KR); Dong-Ho KIM, Goysang-si (KR)

(21) Appl. No.: 19/070,733

(22) Filed: Mar. 5, 2025

(30) Foreign Application Priority Data
Mar. 6, 2024 (KR) 10-2024-0032042

(51) Int. Cl. H04W 4/90; G06Q 50/22; G06Q 50/26; G16H 10/60

(52) U.S. Cl. CPC H04W 4/90; G06Q 50/22; G06Q 50/265; G16H 10/60

(57) ABSTRACT

A method and apparatus for providing a safety care service using artificial intelligence are disclosed. According to an embodiment of the present disclosure, a method for providing a safety care service comprises preprocessing monitoring information of a safety zone obtained from IoT sensors, extracting a situation feature of a current situation in the safety zone by using a situation feature extraction model and the monitoring information, detecting an abnormal situation by using an abnormal situation detection model and the situation feature, receiving a spoken voice from the IoT sensors when an abnormal situation is detected, determining whether the abnormal situation is a caution situation or an emergency situation by using the spoken voice and abnormal situation determination model and taking a countermeasure based on the abnormal situation.

### CROSS-REFERENCE TO RELATED APPLICATIONS

The present application is based on, and claims priority from, Korean Patent Application No. 10-2024-0032042 filed on Mar. 6, 2024, the disclosure of which is incorporated by reference herein in its entirety.

### BACKGROUND OF THE INVENTION

### Technical Field

The present invention relates to a method, apparatus, and system for providing a safety care service using artificial intelligence. More particularly, it relates to a method and apparatus for monitoring a comfort zone using IoT sensors and detecting an abnormal situation to provide countermeasures.

### Background Art

The content described below merely provides background information related to this embodiment, and does not constitute the related art.

Artificial intelligence (AI) is one of the detailed fields of computer science for artificially implementing human learning ability, reasoning ability, perceptual ability, and the like. An artificial intelligence system is a computer system that implements human-level intelligence, and unlike existing rule-based smart systems, it is a system in which machines learn and judge themselves. Existing rule-based smart systems are gradually being replaced by deep learning-based artificial intelligence systems.

In recent years, such artificial intelligence systems have been used to provide safety care service to users in certain areas. Circumstances such as disaster occurrences, people's health problems, or burglary intrusions may occur at any time in people's living spaces such as houses, schools, and the like. In particular, when such a situation occurs, it is difficult to cope with the situation without people around, and thus a situation detection and response system is required to prevent the situation. Therefore, it is necessary to detect a situation such as disaster occurrence, human health abnormality, school violence, or robbery intrusion in a specific area by using artificial intelligence and respond to the situation.

### DISCLOSURE OF INVENTION

### Technical Problem

An object of the present disclosure is to detect whether a current situation is an abnormal situation in a specific area, classify an emergency stage of the abnormal situation, and take countermeasures to solve a problem such as lonely death, school violence, or residential anxiety, which is a social problem.

In addition, according to an embodiment, an object of the present invention is to provide a customized countermeasure for each emergency stage of an abnormal situation.

The problems to be solved by the present invention are not limited to the above-mentioned problems, and other problems that are not mentioned will be clearly understood by those skilled in the art from the following description.

### Solution to Problem

According to the present disclosure, a method for providing a safety care service, the method comprising: preprocessing monitoring information of a safety zone obtained from IoT sensors, extracting a situation feature of a current situation in the safety zone by using a situation feature extraction model and the monitoring information, detecting an abnormal situation by using an abnormal situation detection model and the situation feature, receiving a spoken voice from the IoT sensors when an abnormal situation is detected, and determining whether the abnormal situation is a caution situation or an emergency situation by using the spoken voice and abnormal situation determination model and taking a countermeasure based on the abnormal situation, wherein the taking a countermeasure comprises: transmitting a notification information to a user device by using a caution situation response model when the abnormal situation is determined as the caution situation, and classifying the abnormal situation into a first-stage emergency situation and a second-stage emergency situation when the abnormal situation is determined as the emergency situation, when the abnormal situation is classified as the first-stage emergency situation, transmitting the notification information to the user device by using first-stage emergency situation response model, notifying administrative agency of the abnormal situation, transmitting response confirmation information to the user device to request response confirmation, and receiving emergency situation response information from the user device and when the abnormal situation is classified as the second-stage emergency situation, transmitting the notification information to the user device by using second-stage emergency situation response model, notifying administrative agency of the abnormal situation, transmitting the response confirmation information to the user device to request response confirmation, transmitting administrator movement information to an administrator device to send an administrator to the safety zone, and receiving emergency response information from the administrator device or a device of the visitor when the administrator or a visitor of the administrative agency arrives at the safety zone.

According to the present disclosure, a safety care service providing system comprising: preprocessing unit configured to pre-process monitoring information of a safety zone obtained from IoT sensors, an edge device, configured to extract a situation feature of a current situation in the safety zone by using a situation feature extraction model and the monitoring information, detect an abnormal situation by using an abnormal situation detection model and the situation feature, receive an uttered voice from the IoT sensors when the abnormal situation is detected, determine whether the abnormal situation is a caution situation or an emergency situation by using the uttered voice and an abnormal situation determination model, and transmit notification information to a user device by using a caution situation response model when the abnormal situation is determined as the caution situation and a central server configured to classify the abnormal situation into a first-stage emergency and a second-stage emergency situation when it is determined that the abnormal situation is the emergency situation, transmit the notification information to the user device by using a first-stage emergencies situation response model when the abnormal situation is classified into the first-stage emergency situation, notify an administrative agency of the abnormal situation, transmit response confirmation information to the user device to request a response confirmation, receive emergency response information from the user device, transmit the notification information to the user device by using a second-stage emergency situation response model when the abnormal situation is classified as the second-stage emergency situation, notify an administrative agency of the abnormal situation, transmit the response confirmation information to the user device to request a response confirmation, transmit administrator movement information to administrator device to send an administrator to the safety zone, and receive emergency situation response information from the administrator device or a device of the visitor when the administrator or a visitor of the administrative agency arrives at the safety zone.

A computer-readable recording medium according to the present disclosure, computer-readable recording medium having stored thereon instructions that, wherein the command, when executed by the computer, causes the computer to comprise: preprocessing monitoring information of a safety zone obtained from IoT sensors, extracting a situation feature of a current situation in the safety zone by using a situation feature extraction model and the monitoring information, detecting an abnormal situation by using an abnormal situation detection model and the situation feature, receiving a spoken voice from the IoT sensors when an abnormal situation is detected, and determining whether the abnormal situation is a caution situation or an emergency situation by using the spoken voice and abnormal situation determination model and taking a countermeasure based on the abnormal situation, wherein the taking a countermeasure comprises: transmitting a notification information to a user device by using a caution situation response model when the abnormal situation is determined as the caution situation, and classifying the abnormal situation into a first-stage emergency situation and a second-stage emergency situation when the abnormal situation is determined as the emergency situation, when the abnormal situation is classified as the first-stage emergency situation, transmitting the notification information to the user device by using first-stage emergency situation response model, notifying administrative agency of the abnormal situation, transmitting response confirmation information to the user device to request response confirmation, and receiving emergency situation response information from the user device and when the abnormal situation is classified as the second-stage emergency situation, transmitting the notification information to the user device by using second-stage emergency situation response model, notifying administrative agency of the abnormal situation, transmitting the response confirmation information to the user device to request response confirmation, transmitting administrator movement information to an administrator device to send an administrator to the safety zone, and receiving emergency response information from the administrator device or a device of the visitor when the administrator or a visitor of the administrative agency arrives at the safety zone.

### Effects of the Invention

According to the present disclosure, it is possible to detect whether a current situation is an abnormal situation in a specific area, classify an emergency stage of the abnormal situation, and take countermeasures, thereby reducing human loss and material loss due to social problems.

In addition, according to an embodiment, there is an effect of taking a quick and efficient accident prevention response by taking a customized countermeasure for each emergency stage of an abnormal situation.

Effects that may be obtained in the present disclosure are not limited to the above-mentioned effects, and other effects that are not mentioned will be clearly understood by those skilled in the art from the following description.

### BRIEF DESCRIPTION OF THE DRAWINGS

FIG. 1 is a block diagram for illustrating a system for providing a safety care service, according to an embodiment of the present disclosure.

FIG. 2 is a block diagram for illustrating an edge device according to an embodiment of the present disclosure.

FIG. 3 is a block diagram for illustrating a central server according to an embodiment of the present disclosure.

FIG. 4 is a flowchart for illustrating a process of providing a safety care service, according to an embodiment of the present disclosure.

### DESCRIPTION OF THE PREFERRED EMBODIMENTS OF THE INVENTION

Hereinafter, some exemplary embodiments of the present disclosure will be described in detail with reference to the accompanying drawings. In the following description, like reference numerals preferably designate like elements, although the elements are shown in different drawings. Further, in the following description of some embodiments, a detailed description of known functions and configurations incorporated therein will be omitted for the purpose of clarity and for brevity.

Additionally, various terms such as first, second, A, B, (a), (b), etc., are used solely to differentiate one component from the other but not to imply or suggest the substances, order, or sequence of the components. Throughout this specification, when a part ‘includes’ or ‘comprises’ a component, the part is meant to further include other components, not to exclude thereof unless specifically stated to the contrary. The terms such as ‘unit’, ‘module’, and the like refer to one or more units for processing at least one function or operation, which may be implemented by hardware, software, or a combination thereof.

The following detailed description, together with the accompanying drawings, is intended to describe exemplary embodiments of the present disclosure, and is not intended to represent the only embodiments in which the present disclosure may be practiced.

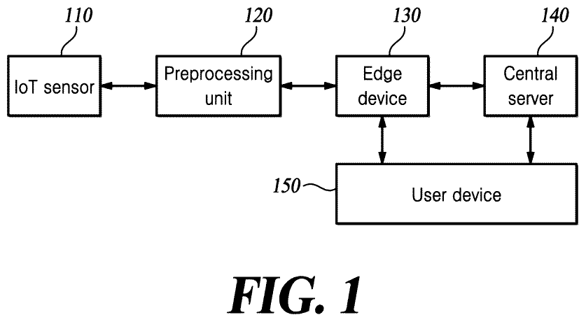

FIG. 1 is a block diagram for illustrating a system for providing a safety care service, according to an embodiment of the present disclosure.

Referring to FIG. 1, a system for providing a safety care service includes all or some of an IoT sensor 110, a preprocessing unit 120, an edge device 130, a central server 140, a user device 150, and the like. The system for providing a safety care service and each component thereof may be implemented by hardware or software, or may be implemented by a combination of hardware and software. In addition, functions of each component may be implemented in software, and one or more processors may be implemented to execute functions of software corresponding to each component.

The IoT sensor 110 monitors the safety zone to obtain monitoring information for detecting and responding to an emergency in the safety zone. Here, the safe area may be an area such as a restroom, a living room, a room, and an entrance door of a detached house, a multi-unit house, an officetel, an apartment, or the like, or an area such as the restroom, a nursery, a gymnasium, a rest area, a classroom, an entrance door, a CCTV blind spot, a staircase, a hallway, and a fence of a school. The monitoring information may include image information, sound information, bio-signal information, and the like. The image information may include information about an image of the safety zone, information about a thermal image of the safety zones, information about images of persons in the safety zones, and information about thermal images of persons in safety zones. The sound information may include information about a sound generated from the safety zone, information about voices of persons in the safety zone, and the like. For example, the sound generated from the safety zone may be a breaking sound, an impact sound, a collision sound, or the like. The voice of persons in the safety zone may be a screaming sound, a rescue request sound, or the like. The biosignal information may include information about breathing, heart rate, body temperature, and the like of persons in the comfort zone.

There may be a plurality of IoT sensors 110. The IoT sensor 110 may include a camera, a complementary metal oxide semiconductor (CMOS)-based thermal imaging camera, a radar, a lidar, a temperature and humidity sensor, an acceleration sensor, an ultrasonic sensor, an infrared sensor, a fire detection sensor, a thermal image detection sensor, a bio-signal detection sensor, an emergency call detection sensor, and the like. The IoT sensor 110 transmits the monitoring information to the preprocessing unit 120.

The preprocessing unit 120 preprocesses the monitoring information received from the IoT sensor 110. The preprocessing unit 120 may remove noise from the monitoring information, extract a region of interest of the monitoring information, divide an image of the monitoring information and normalize the monitoring information. The preprocessing unit 120 transmits the pre-processed monitoring information to the edge device 130.

The edge device 130 extracts the context feature by using the preprocessed monitoring information. The edge device 130 detects an abnormal situation in the safety zone by using the situation feature. When an abnormal situation is detected, the edge device 130 determines whether the current situation is a caution situation or an emergency situation. When the current situation is determined as the caution situation, the edge device 130 may primarily transmit the notification information to the user device 150. The notification information may be information for notifying that the caution situation or an emergency situation has occurred in the safety zone. The edge device 130 may include a long term evolution (LTE) communication module for transmitting notification information. The notification information may be transmitted to the user device 150 by using a large language model (LLM) based chatbot or a text message. When it is determined that the current situation is an emergency situation, the edge device 130 may transmit preprocessed monitoring information and information about an abnormal situation to the central server 140. The information about the abnormal situation may include information about a time at which the abnormal situation is detected, information about a place at which the abnormal situation is detected in the safety zone, and information about a type of the abnormal situation.

The central server 140 may classify the emergency situation into a first-stage emergency situation and a second-stage emergency situation by using the preprocessed monitoring information and the information on the abnormal situation. When the emergency situation is classified as the first-stage emergency situation, the central server 140 may transmit notification information to the user device 150, notify the administrative agency that the emergency situation has occurred, and transmit the response confirmation information to the user device 150 to request the response confirmation. When the emergency situation is classified as the second-stage emergency situation, the central server 140 may transmit notification information to the user device 150, notify the administrative agency that the emergency situation has occurred, transmit response confirmation information to the user device 150 to request response confirmation of the emergency situation, and transmit manager movement information to the manager device to request the manager of the safety zone to visit the safety zone. The central server 140 may manage a history of abnormal situations detected in the safety zone by using the information about the abnormal situations.

The user device 150 may be a smartphone, a smartwatch, a notebook, a digital broadcasting device, a personal digital assistants (PDA), a portable multimedia player (PMP), navigation, a slate PC, a tablet PC, an ultrabook, a wearable device, or the like. The user device 150 may be a device of user registered with the central server 140 and a device of person other than the users registered with the center server 140 in the safety zone. The users registered with the central server 140 may be persons to be kept in the safety zone. The user device 150 may be a preset device.

The IoT sensor 110, the preprocessing unit 120, the edge device 130, the central server 140, and the user device 150 may be connected through at least one wired or wireless communication network. Such wired and wireless communication networks may include wired Internet, wireless Internet, ultra wide band (UWB), long term evolution (LTE), long range (LoRa) wireless communication, Bluetooth, and Zigbee. The wired and wireless communication networks may be grouped according to network usage environments to which the IoT sensor 110, the preprocessing unit 120, the edge device 130, the central server 140, and the user device 150 are connected. The IoT sensor 110, the preprocessing unit 120, the edge device 130, the central server 140, and the user device 150 may be independently connected to and managed by at least one wired/wireless communication network. A connection priority and a connection time for each of the wired and wireless communication networks may be dynamically managed based on a size of a network group, a data usage of each group, and a data transmission cycle.

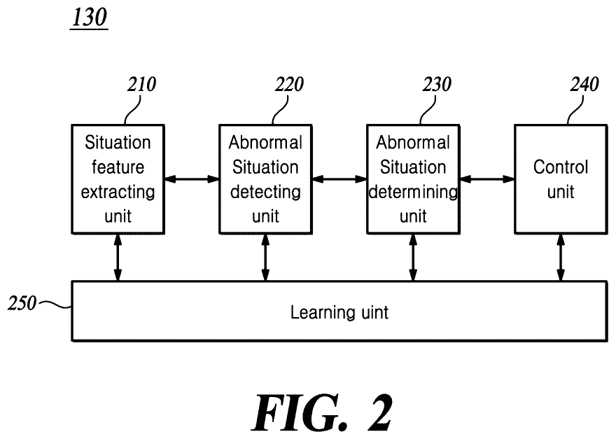

FIG. 2 is a block diagram for illustrating an edge device according to an embodiment of the present disclosure.

Referring to FIG. 2, the edge device 130 includes, in whole or in part, a situation feature extracting unit 210, an abnormal situation detecting unit 220, an abnormal situation determining unit 230, a control unit 240, a learning model 250, and the like. The edge device 130 and each component thereof may be implemented by hardware or software, or may be implemented by a combination of hardware and software. In addition, functions of each component may be implemented in software, and one or more processors may be implemented to execute functions of software corresponding to each component.

The situation feature extracting unit 210 extracts a situation feature of the safety zone by using the monitoring information. The situation feature of the safety zone may be the appearance or wandering of a stranger, a disaster occurrence element of the safety zone, a health abnormality signal of persons registered in the central server 140, and the like. The situation feature extracting unit 210 extracts a situation feature of the safety zone and transmits the extracted situation feature to the abnormal situation detecting unit 220.

The abnormal situation detecting unit 220 detects an abnormal situation using a situation feature of the safety zone. The abnormal situation detecting unit 220 may detect whether a thief, a stalker, or a robber intrudes into the safety zone, whether a disaster occurs in the safety zone, and whether a health abnormality occurs in persons registered in the central server 140. For example, the abnormal situation detecting unit 220 may detect whether a thief, a stalker, or a robber breaks into the safety zone by using clothes, behaviors, moving distances, movement lines, body shapes, and the like of a low-lying person. The abnormal situation detecting unit 220 may detect whether a health abnormality occurs in persons registered in the central server 140 by using a fall, a fall, a low body temperature, a high fever, a respiratory imbalance, unconsciousness, apnea, cardiac arrest, or the like of persons registered in the center server 140. For example, the abnormal situation detecting unit 220 may detect whether a health abnormality occurs in persons registered in the central server 140 by using a fall of persons registered in the center server 140 due to school violence or a dispute in a classroom.

When an abnormal situation is detected, the edge device 130 receives an uttered voice from the IoT sensor 110. When an abnormal situation is detected, the abnormal situation determining unit 230 determines whether the abnormal situation is the caution situation or an emergency situation by using the uttered voice received from the IoT sensor 110. The abnormal situation determining unit 230 may determine whether the abnormal situation is the caution situation or an emergency situation based on whether the abnormal situation may be handled by itself. For example, when a fire occurs in the safety zone, the abnormal situation determining unit 230 may determine the abnormal situation as the caution situation when persons in the safety zone may suppress the fire by themselves, and may determine the abnormal situation as an emergency situation when persons in a safety zone cannot suppress the fire by itself. When a robber breaks into the safety zone, the abnormal situation determining unit 230 may determine the abnormal situation as the caution situation when persons in the safety zone may suppress the robber, and may determine the abnormal situation as an emergency situation when persons in a safety zone cannot suppress the robber. When an abnormality occurs in the health of persons registered in the central server 140 in the safety zone, the abnormal situation determining unit 230 may determine the abnormality as the caution situation when the person having the abnormality or other persons in the safety zone may take medical measures by themselves, and determine the abnormal situation to be an emergency situation when the person with the abnormal situation or the other persons in the safety zone cannot take medical measures by itself.

When the abnormal situation is determined as the caution situation, the control unit 240 may transmit notification information to the user device 150. The control unit 240 may transmit the notification information to devices of persons registered in the central server 140 and devices of persons in the safety zone other than the persons registered in the center server 140. The control unit 240 may preferentially transmit the notification information to devices of persons registered in the central server 140, and then transmit the notification information, to devices of persons in the safety zone other than the persons registered in the center server 140. When the abnormal situation is not an emergency situation but the caution situation, the control unit 240 of the edge device 130 transmits notification information to the user device 150, so that the users may quickly cope with the caution situation.

The learning model 250 may be a deep learning based model. The edge device 130 may further include a learning unit (not shown) for training the learning model 250 in advance. The learning unit may train the learning model 250 in advance using supervised learning, unsupervised learning, semi-supervised learning, reinforcement learning, and/or the like. The learning model 250 may include a situation feature extraction model, an abnormal situation detection model, an abnormal situation determination model, and a caution response model.

The situation feature extracting unit 210 may extract a context feature of the safety zone by using a context feature extraction model. The abnormal situation detecting unit 220 may detect an abnormal situation using an abnormal situation detection model. The abnormal situation determining unit 230 may determine whether the abnormal situation is the caution situation or an emergency situation by using an abnormal situation determination model. The control unit 240 may transmit the notification information to the user device 150 by using the caution response model.

The learning unit may learn the situation feature extraction model, the abnormal situation detection model, the abnormal situation determination model, and the caution response model by using the preprocessed monitoring information, the situation feature information, the abnormal situation information, and the like in the edge device 130 as learning data. The learning unit may perform joint learning by using the parameters received from the central server 140 to learn the situation feature extraction model, the abnormal situation detection model, the abnormal situation determination model, and the caution response model. The situation feature extraction model, the abnormal situation detection model, the abnormal situation determination model, and the caution response model may be updated by using an SGD (Stochastic Gradient Descent) algorithm to minimize a loss value of a loss function.

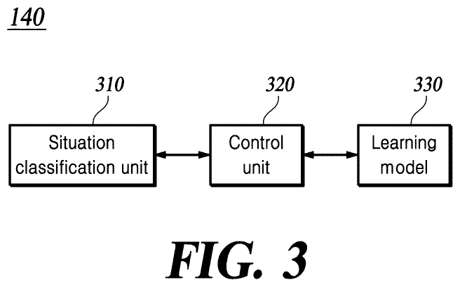

FIG. 3 is a block diagram for illustrating a central server according to an embodiment of the present disclosure.

Referring to FIG. 3, the central server 140 includes, in whole or in part, a situation classification unit 310, a control unit 320, a learning model 330, and the like. The central server 140 and each component thereof may be implemented by hardware or software, or may be implemented by a combination of hardware and software. In addition, functions of each component may be implemented in software, and one or more processors may be implemented to execute functions of software corresponding to each component.

When the edge device 130 determines an abnormal situation as an emergency situation, the situation classification unit 310 may determine the severity of the emergency situation by using preprocessed monitoring information, information on situation features, information on abnormal situations, and the like. The situation classification unit 310 classifies the emergency situation into a first-stage emergency situation and the second-stage emergency situation by using the severity of the emergency situation.

For example, when an emergency situation is a situation in which a fire has occurred in a safety zone and persons in the safety zone cannot suppress the fire, the situation classification unit 310 may classify the emergency situation as a first-stage emergency situation. When an emergency situation is a situation in which a robber intrudes into the safety zone and persons cannot suppress the robber in the safety zone, the situation classification unit 310 may classify the emergency situation as a first-stage emergency situation. When the emergency situation is a situation in which a person registered in the central server 140 in the safety zone is unconscious, the situation classification unit 310 may classify the emergency situation as the second-stage emergency situation. When the emergency situation is a situation in which a robber intrudes into the safety zone and the robber injures persons, the situation classification unit 310 may classify the emergency situation as the second-stage emergency situation. When the emergency situation is a situation in which a large-scale fire has occurred in the safety zone and persons in the safety zone are injured due to the fire, the situation classification unit 310 may classify the emergency situation as the second-stage emergency situation.

The control unit 320 may take countermeasures for the first-stage emergency situation and the second-stage emergency situation, respectively. When the emergency situation is classified as the first-stage emergency situation, the control unit 320 may transmit notification information to the user device 150, notify the administrative agency that the emergency situation has occurred, and transmit the response confirmation information to the user device 150 to request the response confirmation. The correspondence confirmation information may be information requesting confirmation of a correspondence to an emergency. The control unit 320 may receive emergency response information from the user device 150. The emergency response information may be information about a current state after responding to an emergency. The control unit 320 may preferentially transmit the correspondence information to the devices of the persons registered in the central server 140, and then transmit the corresponding correspondence information to devices of persons in the safety zone other than the persons registered in a central server 140. However, when the control unit 320 receives the emergency response information after transmitting the response confirmation information to the devices of the persons registered in the central server 140, the control unit 320 may not transmit the response confirmation information from the devices of persons in the safety zone other than the persons registered in a central server 140.

When the emergency situation is classified as the second-stage emergency situation, the control unit 320 may transmit notification information to the user device 150, notify the administrative agency that the emergency situation has occurred, transmit response confirmation information to the user device 150 to request response confirmation, and transmit manager movement information to the manager device to request the manager to visit the safety zone. Here, the manager may be a person who manages the safety zone. The administrator moving information may be information about a person who manages the safety zone. For example, when the robber enters the safety zone and injures persons, the administrator may visit the safety zone, check the situation, and notify the central server 140 that the police are confronting the robber by using the user device 150. When a large-scale fire occurs in the safety zone, the manager may visit the safety zone to check the situation and notify the central server 140 that the fire fighter arrives and extinguishes the fire.

When the visitor of the manager or the administrative agency arrives at the safety zone, the control unit 320 may receive emergency response information from the device of the manager or from the device of a visitor of the administrative agency. The emergency response information may be information about a current state after responding to an emergency.

The learning model 330 may be a deep learning based model. The central server 140 may further include a learning unit (not shown) for training the learning model 330 in advance. The learning unit may train the learning model 330 in advance by using supervised learning, unsupervised learning, semi-supervised learning, reinforcement learning, and/or the like. The learning model 330 may include a first-stage emergency situation response model and a second-stage emergency situation response models. The control unit 320 may take a countermeasure against the first-stage emergency situation and the second-stage emergency situation by using a first-stage emergency situation response model and the second-stage emergency situation response models.

The learning unit may learn the first-stage emergency situation response model and the second-stage emergency situation response models by using the preprocessed monitoring information, the situation feature information, the abnormal situation information, and the like in the central server 140 as learning data. The learning unit may generate a global parameter by averaging parameters received from the edge device 130. The learning unit may perform joint learning by using the global parameter to learn the first-stage emergency situation response model and the second-stage emergency situation response models. The first-stage emergency situation response model and the second-stage emergency situation response models may include a hidden markov model.

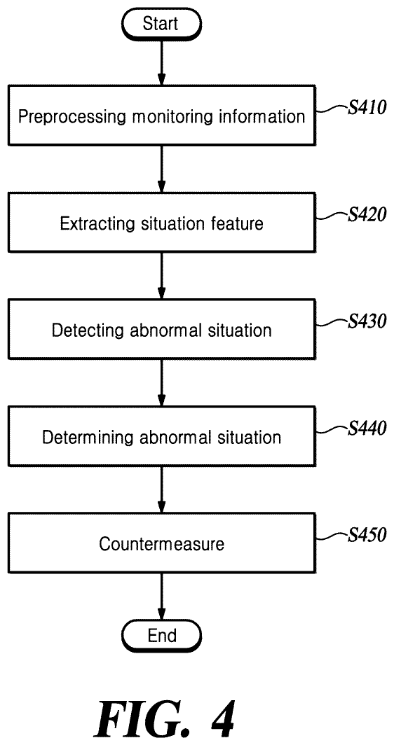

FIG. 4 is a flowchart for illustrating a process of providing a safety care service, according to an embodiment of the present disclosure.

Referring to FIG. 4, the preprocessing unit 120 preprocesses the monitoring information of the safety zone obtained from the IoT sensor 110 (S410). The situation feature extracting unit 210 extracts the situation feature of the current situation in the safety zone by using the situation feature extraction model and the monitoring information (S420). The abnormal situation detecting unit 220 detects the abnormal situation using the abnormal situation detection model and the situation feature (S430). When an abnormal situation is detected, the edge device 130 may receive an uttered voice from the IoT sensor 110. The abnormal situation determining unit 230 determines whether the abnormal situation is the caution situation or an emergency situation by using the uttered voice and the abnormal situation determination model (S440). The control unit issues a countermeasure based on the abnormal situation (S450).

When the abnormal situation is determined as the caution situation, the control unit 240 may transmit the notification information to the user device 150 by using the caution situation response model. When the abnormal situation is determined to be an emergency situation, the situation classification unit 310 may classify the abnormal situation into a first-stage emergency situation and the second-stage emergency situation. When the abnormal situation is classified as a first-stage emergency situation, the control unit 320 may transmit notification information to the user device by using the first-stage emergency situation response model, notify the administrative agency of the abnormal situation, transmit response confirmation information to the user device 150 to request response confirmation, and receive emergency response information from the user device 150. When the abnormal situation is classified as the second-stage emergency, the control unit 320 may transmit notification information to the user device 150 by using the second-stage emergency situation response model, notify the administrative agency of the abnormal situation, transmit the response confirmation information to the user device 150 to request the response confirmation, transmit the administrator movement information to the administrator device to send the administrator to the safety zone, and receive the emergency response information from the device of the administrator device or the visitor of the administrative agency when the administrator or the visitor arrives at the safety zone.

Each element of the apparatus or method in accordance with the present invention may be implemented in hardware or software, or a combination of hardware and software. The functions of the respective elements may be implemented in software, and a microprocessor may be implemented to execute the software functions corresponding to the respective elements.

Various embodiments of systems and techniques described herein can be realized with digital electronic circuits, integrated circuits, field programmable gate arrays (FPGAs), application specific integrated circuits (ASICs), computer hardware, firmware, software, and/or combinations thereof. The various embodiments can include implementation with one or more computer programs that are executable on a programmable system. The programmable system includes at least one programmable processor, which may be a special purpose processor or a general purpose processor, coupled to receive and transmit data and instructions from and to a storage system, at least one input device, and at least one output device. Computer programs (also known as programs, software, software applications, or code) include instructions for a programmable processor and are stored in a “computer-readable recording medium.”

The computer-readable recording medium may include all types of storage devices on which computer-readable data can be stored. The computer-readable recording medium may be a non-volatile or non-transitory medium such as a read-only memory (ROM), a random access memory (RAM), a compact disc ROM (CD-ROM), magnetic tape, a floppy disk, or an optical data storage device. In addition, the computer-readable recording medium may further include a transitory medium such as a data transmission medium. Furthermore, the computer-readable recording medium may be distributed over computer systems connected through a network, and computer-readable program code can be stored and executed in a distributive manner.

Although operations are illustrated in the flowcharts/timing charts in this specification as being sequentially performed, this is merely an exemplary description of the technical idea of one embodiment of the present disclosure. In other words, those skilled in the art to which one embodiment of the present disclosure belongs may appreciate that various modifications and changes can be made without departing from essential features of an embodiment of the present disclosure, that is, the sequence illustrated in the flowcharts/timing charts can be changed and one or more operations of the operations can be performed in parallel. Thus, flowcharts/timing charts are not limited to the temporal order.

Although exemplary embodiments of the present disclosure have been described for illustrative purposes, those skilled in the art will appreciate that various modifications, additions, and substitutions are possible, without departing from the idea and scope of the claimed invention. Therefore, exemplary embodiments of the present disclosure have been described for the sake of brevity and clarity. The scope of the technical idea of the present embodiments is not limited by the illustrations. Accordingly, one of ordinary skill would understand that the scope of the claimed invention is not to be limited by the above explicitly described embodiments but by the claims and equivalents thereof.

What is claimed is:

1. A method performed by a system for providing safety care service, the method comprising:
preprocessing monitoring information of a safety zone obtained from IoT sensors;
extracting a situation feature of a current situation in the safety zone by using a situation feature extraction model and the monitoring information;
detecting an abnormal situation by using an abnormal situation detection model and the situation feature;
receiving a spoken voice from the IoT sensors when an abnormal situation is detected, and determining whether the abnormal situation is a caution situation or an emergency situation by using the spoken voice and abnormal situation determination model; and
taking a countermeasure based on the abnormal situation,
wherein the taking a countermeasure comprises:
transmitting a notification information to a user device by using a caution situation response model when the abnormal situation is determined as the caution situation, and classifying the abnormal situation into a first-stage emergency situation and a second-stage emergency situation when the abnormal situation is determined as the emergency situation;
when the abnormal situation is classified as the first-stage emergency situation, transmitting the notification information to the user device by using first-stage emergency situation response model, notifying administrative agency of the abnormal situation, transmitting response confirmation information to the user device to request response confirmation, and receiving emergency situation response information from the user device; and
when the abnormal situation is classified as the second-stage emergency situation, transmitting the notification information to the user device by using second-stage emergency situation response model, notifying administrative agency of the abnormal situation, transmitting the response confirmation information to the user device to request response confirmation, transmitting administrator movement information to an administrator device to send an administrator to the safety zone, and receiving emergency response information from the administrator device or a device of the visitor when the administrator or a visitor of the administrative agency arrives at the safety zone.

2. The method of claim 1, wherein:
the monitoring information comprises image information, sound information, and biosignal information,
the image information comprises information about images capturing safety zone, information about thermal images capturing safety zone, information about images capturing persons within safety zone, and information about thermal images capturing persons within safety zone,
the sound information comprises information about sound generated from safety zone and information about voices of persons within safety zone, and
the biosignal information comprises information about respiration, information about heart rate, and information about body temperature, of persons within safety zone.

3. The method of claim 1, wherein:
the notification information is transmitted in the order of devices of users registered in a central server and devices of persons within the safety zone who are not registered in the central server; and
the notification information is transmitted using a large language model (LLM) based chatbot or a text message.

4. The method of claim 1, wherein:
the situation feature extraction model, the abnormal situation detection model and the abnormal situation determination model, and the caution situation response model are learned by federated learning by using parameters received from a central server; and
the situation feature extraction model, the abnormal situation detection model and the abnormal situation determination model, and the caution situation response model are updated by using a stochastic gradient descent (SGD) algorithm.

5. The method of claim 1, wherein:
the first-stage emergency situation response model and the second-stage emergency situation response model are learned by federated learning by using a global parameter obtained by averaging parameters received from an edge device; and
the first-stage emergency situation response model and the second-stage emergency situation response models comprise a Hidden Markov model.

6. A system for providing safety care service, comprising:
preprocessing unit configured to pre-process monitoring information of a safety zone obtained from IoT sensors;
an edge device, configured to extract a situation feature of a current situation in the safety zone by using a situation feature extraction model and the monitoring information, detect an abnormal situation by using an abnormal situation detection model and the situation feature, receive an uttered voice from the IoT sensors when the abnormal situation is detected, determine whether the abnormal situation is a caution situation or an emergency situation by using the uttered voice and an abnormal situation determination model, and transmit notification information to a user device by using a caution situation response model when the abnormal situation is determined as the caution situation; and
a central server configured to classify the abnormal situation into a first-stage emergency and a second-stage emergency situation when it is determined that the abnormal situation is the emergency situation, transmit the notification information to the user device by using a first-stage emergencies situation response model when the abnormal situation is classified into the first-stage emergency situation, notify an administrative agency of the abnormal situation, transmit response confirmation information to the user device to request a response confirmation, receive emergency response information from the user device, transmit the notification information to the user device by using a second-stage emergency situation response model when the abnormal situation is classified as the second-stage emergency situation, notify an administrative agency of the abnormal situation, transmit the response confirmation information to the user device to request a response confirmation, transmit administrator movement information to administrator device to send an administrator to the safety zone, and receive emergency situation response information from the administrator device or a device of the visitor when the administrator or a visitor of the administrative agency arrives at the safety zone.

7. The system of claim 6, wherein:
the monitoring information comprises image information, sound information, and biosignal information,
the image information comprises information about images capturing safety zone, information about thermal images capturing safety zone, information about images capturing persons within safety zone, and information about thermal images capturing persons within safety zone,
the sound information comprises information about sound generated from safety zone and information about voices of persons within the safety zone, and
the biosignal information comprises information about respiration, information about heart rate, and information about body temperature, of persons within safety zone.

8. The system of claim 6, wherein:
the notification information is transmitted in the order of devices of users registered in a central server and devices of persons within the safety zone who are not registered in the central server; and
the notification information is transmitted using a large language model (LLM) based chatbot or a text message.

9. The system of claim 6, wherein:
the situation feature extraction model, the abnormal situation detection model and the abnormal situation determination model, and the caution situation response model are learned by federated learning by using parameters received from the central server; and
wherein the situation feature extraction model, the abnormal situation detection model and the abnormal situation determination model, and the caution situation response model are updated by using an SGD algorithm.

10. The system of claim 6, comprising:
the first-stage emergency situation response model and the second-stage emergency situation response model are learned by federated learning by using a global parameter obtained by averaging parameters received from an edge device; and
the first-stage emergency situation response model and the second-stage emergency situation response model comprise a hidden Markov model.

11. A computer-readable recording medium having stored thereon instructions that, wherein the command, when executed by the computer, causes the computer to execute:
preprocessing monitoring information of a safety zone obtained from IoT sensors;
extracting a situation feature of a current situation in the safety zone by using a situation feature extraction model and the monitoring information;
detecting an abnormal situation by using an abnormal situation detection model and the situation feature;
receiving a spoken voice from the IoT sensors when an abnormal situation is detected, and determining whether the abnormal situation is a caution situation or an emergency situation by using the spoken voice and abnormal situation determination model; and
taking a countermeasure based on the abnormal situation,
wherein the taking a countermeasure comprises:
transmitting a notification information to a user device by using a caution situation response model when the abnormal situation is determined as the caution situation, and classifying the abnormal situation into a first-stage emergency situation and a second-stage emergency situation when the abnormal situation is determined as the emergency situation;
when the abnormal situation is classified as the first-stage emergency situation, transmitting the notification information to the user device by using first-stage emergency situation response model, notifying administrative agency of the abnormal situation, transmitting response confirmation information to the user device to request response confirmation, and receiving emergency situation response information from the user device; and
when the abnormal situation is classified as the second-stage emergency situation, transmitting the notification information to the user device by using second-stage emergency situation response model, notifying administrative agency of the abnormal situation, transmitting the response confirmation information to the user device to request response confirmation, transmitting administrator movement information to an administrator device to send an administrator to the safety zone, and receiving emergency response information from the administrator device or a device of the visitor when the administrator or a visitor of the administrative agency arrives at the safety zone
